(19) United States
(12) Patent Application Publication
(10) Pub. No.: US 2025/0001116 A1
(43) Pub. Date: Jan. 2, 2025

(54) CAPNOGRAPHY CANNULAS

(71) Applicant: Oridion Medical 1987 Ltd., Jerusalem (IL)

(72) Inventors: Denis Glozman, Modiin (IL); Alexander Rabkin, Mevaseret Zion (IL); Shai Fleischer, Modiin (IL); Alon Sasson, Rehovot (IL); Doron Lilo, Rishon - LeZiyyon (IL); Avner Bar Lev, Givatayim (IL)

(21) Appl. No.: 18/678,923

(22) Filed: May 30, 2024

Related U.S. Application Data
(60) Provisional application No. 63/510,534, filed on Jun. 27, 2023.

(51) Int. Cl. A61M 16/06

(52) U.S. Cl. CPC A61M 16/0672; A61M 2202/0007; A61M 2202/0208; A61M 2210/0618; A61M 2230/432

(57) ABSTRACT

An example cannula may include a cannula body defining an exhalation lumen configured to receive a volume of exhalation from a patient. The cannula body defines an oxygen inlet at a surface of the cannula body. The cannula body defines a plurality of elongated oxygen delivery openings fluidically coupled to the oxygen inlet and being arranged and sized to deliver substantially symmetrical oxygen flow to nostrils of the patient.

### CROSS-REFERENCE TO RELATED APPLICATION

This application claims priority to and the benefit of U.S. Provisional Patent Application No. 63/510,534, entitled “CAPNOGRAPHY CANNULAS,” filed on Jun. 27, 2023, which is incorporated herein by reference in its entirety for all purposes.

### TECHNICAL FIELD

The present disclosure generally relates to capnography, and cannulas for capnography.

### BACKGROUND

Capnography is used to monitor exhalations from a patient, in particular, to determine carbon dioxide content in respiration by the patient. Carbon dioxide concentration in exhaled breath may differ at different stages of exhalation, and by physiological conditions of the patient. Capnography may be used to determine deviations from nominal exhalation characteristics of a patient. The patient may be intubated or non-intubated. Capnography may use one or more sensors to measure an amount of carbon dioxide in exhalation from the patient, for example, in-line sensors, or sensors in remote monitors.

Capnography cannulas may be used to receive and transport exhalation from a patient to a capnography analysis module, and to deliver oxygen to the patient.

### SUMMARY

In general, this disclosure describes cannulas for capnography. In some examples, cannulas according to the present disclosure include one or more prongs configured to reduce or prevent mixing of oxygen (being delivered to the patient via the cannula) with exhaled breath. Reducing or preventing such mixing may promote accuracy of capnography readings. In some examples, cannulas according to the present disclosure include a cannula body defining one or more oxygen delivery openings configured to modify the velocity of oxygen from an oxygen source to be supplied to a patient, and distribute oxygen substantially evenly (e.g., substantially symmetrically) with respect to the nostrils of the patient. The oxygen delivery openings may be substantially similar to each other, or may differ in one or both of size or shape. For example, elongated oxygen delivery openings may provide a more even and uniform flow of oxygen in contrast with oxygen delivery openings that are formed as circular holes. In some examples, oxygen delivery openings may be round or circular, and distributed along a surface of the cannula body in a pattern configured to distribute oxygen substantially evenly. The oxygen delivery openings may be fluidically coupled to the oxygen supply by a distribution section defined by the cannula body.

In some examples according to the present disclosure, an example cannula includes a cannula body and at least one prong extending from the cannula body. The cannula body defines an exhalation lumen configured to receive a volume of exhalation from a patient. The at least one prong defines a prong lumen fluidically coupled to the exhalation lumen. The prong lumen extends to a prong opening configured to introduce the volume of exhalation from the patient into the exhalation lumen. At least a portion of the prong opening faces radially outward from the at least one prong.

In some examples according to the present disclosure, an example cannula includes a cannula body defining a body lumen and a prong extending from the cannula body. The prong defines a prong lumen fluidically coupled to the body lumen. The prong defines a side opening open to the prong lumen.

In some examples according to the present disclosure, an example system includes a capnography analysis module and a cannula fluidically coupled to the capnography analysis module and to deliver a volume of exhalation from a patient to the capnography analysis module. The cannula includes a cannula body defining a body lumen and a prong extending from the cannula body. The prong defines a prong lumen fluidically coupled to the body lumen. The prong defines a side opening open to the prong lumen.

In some examples according to the present disclosure, an example cannula includes a cannula body. The cannula body defines an exhalation lumen configured to receive a volume of exhalation from a patient. The cannula body defines an oxygen inlet at a surface of the cannula body. The cannula body defines at least one elongated oxygen delivery opening configured to deliver oxygen to the patient. The at least one elongated oxygen delivery opening extends along a surface of the cannula body and is fluidically coupled to the oxygen inlet by a distribution section defined by the cannula body. The distribution section has a major cross-sectional area greater than a major cross-sectional area of the oxygen inlet.

In some examples according to the present disclosure, an example cannula includes a cannula body. The cannula body defines an exhalation lumen configured to receive a volume of exhalation from a patient. The cannula body defines an oxygen inlet at a surface of the cannula body. The cannula body defines a plurality of elongated oxygen delivery openings fluidically coupled to the oxygen inlet and being arranged and sized to deliver substantially symmetrical oxygen flow to nostrils of the patient.

In some examples according to the present disclosure, an example system includes a capnography analysis module, an oxygen supply, and a cannula including a cannula body. The cannula body defines an oxygen inlet at a surface of the cannula body. The cannula body defines a plurality of elongated oxygen delivery openings fluidically coupled to the oxygen inlet and being arranged and sized to deliver substantially symmetrical oxygen flow to nostrils of the patient.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF THE DRAWINGS

Advantages of the disclosed techniques may become apparent upon reading the following detailed description and upon reference to the drawings in which:

FIG. 1 is a block diagram of an embodiment of a capnography system including a cannula with a cannula body configured to transport a volume of exhalation to a capnography analysis module;

FIG. 2A is a rear perspective view of an embodiment of a cannula including a cannula body and at least one prong extending from the cannula body and configured to receive a volume of exhalation from a patient;

FIG. 2B is a partial perspective view of the at least one prong of the cannula of FIG. 2A;

FIG. 3 is a rear perspective view of an embodiment of a cannula including a cannula body, at least one prong, and at least one oxygen delivery opening;

FIG. 4 is a partial perspective view of an embodiment of a prong defining a recessed prong opening extending to an open prong end;

FIG. 5 is a partial perspective view of an embodiment of a prong defining a prong opening extending to an open prong end;

FIG. 6 is a partial perspective view of an embodiment of a prong defining an inclined prong opening;

FIG. 7 is a partial perspective view of an embodiment of a prong including a pair of opposing prong edges extending radially away from each other;

FIG. 8A is a perspective view of an embodiment of a cannula including a cannula body and an elongated oxygen delivery opening;

FIG. 8B is a top perspective view of the cannula of FIG. 8A;

FIG. 8C is perspective view of the cannula of FIG. 8A showing an exhalation lumen configured to receive a volume of exhalation from a patient;

FIG. 9 is a perspective view of an embodiment of a cannula including a cannula body defining a pair of elongated oxygen delivery openings coupled to an oxygen inlet;

FIG. 10 is a top perspective view of an embodiment of a cannula including a cannula body defining an elongated oxygen delivery opening having a rectangular periphery extending between a pair of prongs;

FIG. 11 is a top perspective view of an embodiment of a cannula including a cannula body defining an elongated oxygen delivery opening between and spaced from a pair of prongs;

FIG. 12 is a top perspective view of an embodiment of a cannula including a cannula body defining an elongated oxygen delivery opening extending from a distribution section including an angled wall and a flat wall;

FIG. 13 is a top perspective view of an embodiment of a cannula including a cannula body defining an elongated oxygen delivery opening extending from a distribution section including a pair of opposed angled walls;

FIG. 14 is a front schematic view of a contour of a distribution section including a triangular wall;

FIG. 15 is a front schematic view of a contour of a distribution section having a rectangular wall;

FIG. 16 is a front schematic view of a contour of a distribution section having a boat-shaped wall;

FIG. 17A is a perspective view of an embodiment of a cannula including a cannula body defining an elongated oxygen delivery opening extending from a distribution section coupled to a side oxygen inlet;

FIG. 17B is a top perspective view of the cannula of FIG. 17A;

FIG. 18A is a perspective view of an embodiment of a cannula including a rounded cannula body defining an elongated oxygen delivery opening extending from a distribution section coupled to a side oxygen inlet;

FIG. 18B is a top perspective view of the cannula of FIG. 18A;

FIG. 19A is a perspective view of an embodiment of a cannula including a cannula body defining an elongated oxygen delivery opening extending from a distribution section coupled to a side oxygen inlet by a plurality of inlet lumens;

FIG. 19B is a top perspective view of the cannula of FIG. 19A;

FIG. 20 is a perspective view of an embodiment of a cannula including a cannula body defining an elongated oxygen delivery opening extending from a distribution section coupled to a side oxygen inlet at a location laterally offset from a center of the distribution section;

FIG. 21 is a perspective view of an embodiment of a cannula including a cannula body defining an elongated oxygen delivery opening extending from a wedge-shaped distribution section;

FIG. 22 is a perspective view of an embodiment of a cannula including a cannula body defining an elongated oxygen delivery opening fluidically coupled to an oxygen inlet by a diverging supply conduit;

FIG. 23 is a perspective view of an embodiment of a cannula including a cannula body defining an elongated asymmetric oxygen delivery opening;

FIG. 24 is a perspective view of an embodiment of a cannula including a cannula body defining one or more distribution sections extending to and fluidly coupled to a plurality of elongated oxygen delivery openings extending along a longitudinal axis of the cannula;

FIG. 25 is a perspective view of an embodiment of a cannula including a cannula body defining one or more distribution sections extending to and fluidly coupled to a trio of elongated oxygen delivery openings extending along a longitudinal axis of the cannula;

FIG. 26 is a perspective view of an embodiment of a cannula including a cannula body defining one or more distribution sections extending to and fluidly coupled to a plurality of elongated oxygen delivery openings extending transverse to a longitudinal axis of the cannula;

FIG. 27 is a perspective view of an embodiment of a cannula including a cannula body defining one or more distribution sections extending to and fluidly coupled to four elongated oxygen delivery openings extending transverse to a longitudinal axis of the cannula;

FIG. 28 is a perspective view of an embodiment of a cannula including a cannula body defining one or more distribution sections extending to and fluidly coupled to a plurality of circular oxygen delivery openings extending along a longitudinal axis of the cannula;

FIG. 29 is a perspective view of an embodiment of a cannula including a cannula body defining one or more distribution sections extending to and fluidly coupled to a trio of circular oxygen delivery openings extending along or with respect to a longitudinal axis of cannula body;

FIG. 30 is a perspective view of an embodiment of a cannula including a cannula body defining one or more distribution sections extending to and fluidly coupled to a plurality of pairs of elongated oxygen delivery openings extending transverse to a longitudinal axis of the cannula;

FIG. 31 is a perspective view of an embodiment of a cannula including a cannula body defining an elongated oxygen delivery opening extending from a distribution section coupled to an oxygen vortex;

FIG. 32 is a perspective view of an embodiment of a cannula including a cannula body defining one or more distribution sections extending to and fluidly coupled to a plurality of pairs of circular oxygen delivery openings extending along or with respect to a longitudinal axis of the cannula;

FIG. 33 is a perspective view of an embodiment of a cannula including a cannula body defining one or more elongated oxygen delivery openings and including a mouthpiece configured to receive exhalation from a patient; and

FIG. 34 is a is a perspective view of an embodiment of a cannula including a cannula body defining one or more circular oxygen delivery openings and including a mouthpiece configured to receive exhalation from a patient.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

The present disclosure describes capnography cannulas. A capnography cannula is configured to receive and transport exhalation from a patient to a capnography analysis module. The cannula includes at least one prong configured to facilitate receiving exhalation from the patient. For example, the prong may define a prong lumen, and may be configured to the placed within a nostril of the patient. In some embodiments, the cannula may further be used to deliver oxygen to the patient.

Non-intubated capnography in general may be performed using nasal or oral/nasal cannulas. In some embodiments, a capnography cannula enables simultaneous or intermittent supply of oxygen or oxygen-enriched gas to the patient while sampling exhalation. In some cases, the flow rate of oxygen being delivered to the patient may be up to 5 liters per minute (LPM). Moderate-to-high oxygen flow may include flow rates of 1 LPM to 60 LPM, such as 2 LPM to 40 LPM, or such as 2 LPM to 15 LPM. When oxygen is supplied to the patient via some existing cannulas, the flow of oxygen or oxygen-rich gas may mix with exhaled breath being sampled or received by the cannula, diluting the original amount of the carbon dioxide and other gases in the exhalation. Such dilution may alter the capnogram significantly and can lead to erroneous readings, for example, of the end-tidal carbon dioxide (EtCO₂) partial pressure, or readings of other gases.

Cannulas according to the present disclosure are configured to provide separation between oxygen inflow and exhalation outflow, thus promoting accuracy of capnography analysis monitoring by reducing or avoiding introduction or mixing of oxygen into exhalation. In some embodiments, an accurate capnography reading is associated with a difference between actual and measured EtCO2 being 4 millimeters of mercury (mmHg) or less, such as 2 mmHg or less.

In some embodiments, the separation between oxygen inflow and exhalation outflow is promoted by a physical barrier (for example, a wall of a prong) between exhalation flow and oxygen flow, and further by redirecting one or both of the exhalation flow or the oxygen flow. Cannulas according to the present disclosure may further promote efficiency and robustness of oxygen delivery, while reducing patient discomfort during oxygen supply, for example, via placement and orientation of oxygen delivery openings on the cannula. Oxygen delivery efficiency may be determined as a ratio between concentration of supplied oxygen and concentration of oxygen in an inhaled gas or gaseous mixture.

In some embodiments, a cannula includes a cannula body and at least one prong extending from the cannula body. The cannula body defines an exhalation lumen configured to receive a volume of exhalation from a patient. The at least one prong defines a prong lumen fluidically coupled to the exhalation lumen. The prong lumen extends to a prong opening configured to introduce the volume of exhalation from the patient into the exhalation lumen. At least a portion of the prong opening faces radially outward from the at least one prong (e.g., radially outwards from a central longitudinal axis of the prong). In contrast with prongs with closed side surfaces and only including distal end openings to receive exhalation, such an orientation and placement of the prong opening of the cannulas described herein may reduce or prevent dilution of exhalation with oxygen received by the patient. Further, prong length, prong direction or orientation relative to a cannula body, and prong opening or exhalation lumen size, shape, location may further facilitate separation between oxygen flow and exhalation, which in turn may promote accuracy of capnography readings.

Variations in one or more prong openings may include locating a prong opening along a side of the prong, facing towards the nasal cavity. The prong opening may be tilted with respect to a central longitudinal axis of the prong (extending from a proximal end closest to the cannula body to a distal end furthest from the canula body), for example, facing towards the inferior turbinate and tilted toward the median sagittal plane of the patient when the cannula is properly positioned on the patient. In some embodiments, the prong opening is round and/or elongated (e.g., having a greater dimension in a length direction, measured along a longitudinal axis of the prong than in a width direction, measured in a direction orthogonal to the longitudinal axis). The prong opening location can be positioned at any suitable location along the prong length (measured along the longitudinal axis). In some embodiments, the prong opening of a particular prong is closer to the distal tip than to the cannula body. In some embodiments, the prong opening includes one or more support structures, for example, a scoop or a shield, to promote collection of exhalation and separation from oxygen flow, which may be directed towards the superior part of the nasal cavity.

Additionally or alternatively, the configuration of prongs, in some embodiments described herein, facilitate separation between oxygen flow and exhalation by the configuration of oxygen delivery openings and/or inlets. For example, the location of the inlet to which the oxygen supply tube is connected, or the size, shape, location, or directionality of oxygen delivery openings along a cannula surface, may be modified to facilitate such separation.

In some embodiments, a cannula includes a cannula body. The cannula body defines an exhalation lumen configured to receive a volume of exhalation from a patient. The cannula body defines an oxygen inlet at a surface of the cannula body. The cannula body defines at least one elongated oxygen delivery opening configured to deliver oxygen to the patient. The at least one elongated oxygen delivery opening extends along a surface of the cannula body and is fluidically coupled to the oxygen inlet by a distribution section defined by the cannula body. The distribution section has a major cross-sectional area greater than a major cross-sectional area of the oxygen inlet. One or more of the size or the shape of the oxygen delivery opening, of the oxygen inlet, or of the distribution section may be configured to promote uniform and symmetric oxygen flow to nostrils of the patient.

In some embodiments, the cannula body may be directly connectable to oxygen tubing. For example, a portion of oxygen tubing may be received within the cannula body, or be coupled to the cannula body. The direct connection may include one or more of slits, round holes, square holes, or holes or slits of any suitable similar or different shapes. Separation between oxygen inflow and exhalation outflow may reduce or prevent the mixing of oxygen supply with breath, including exhaled CO₂, in turn mitigating potential errors in a capnogram arising from mixing of flows. Cannulas according to the present disclosure may also provide symmetry of oxygen flow to the patient, for example, in case of a problem with one of the nostrils of a patient. Cannulas according to the present disclosure may facilitate controlling the directionality and velocity of oxygen flow, to reduce the discomfort for the patients, caused by constant oxygen flow to the nose.

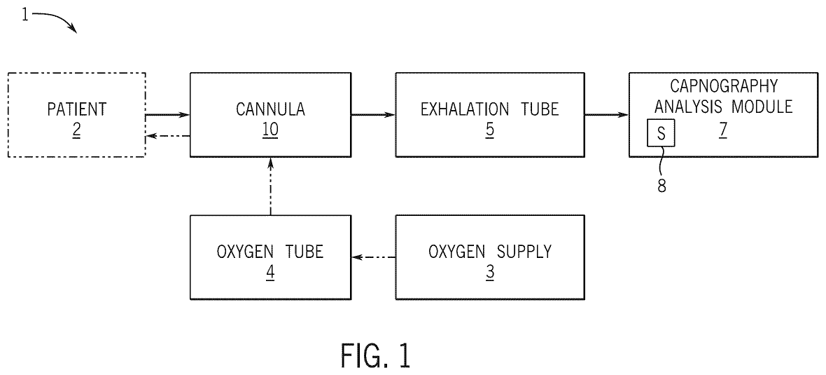

FIG. 1 is a block diagram of an embodiment of a capnography system 1 including a capnography cannula 10 (also referred to as cannula 10). The cannula 10 may be positioned about or within a mouth or nasal cavity of a patient 2 to receive exhalation from the patient 2. The exhalation is transported via an exhalation tube 5 to a capnography analysis module 7. The capnography analysis module 7 may include one or more sensors 8 configured to detect carbon dioxide and/or other gases in the exhalation, and generate one or more signals indicative of carbon dioxide concentration, carbon dioxide amount, or generally a gas composition of a volume of exhalation. The capnography analysis module 7 may be a part of a capnography monitor, or may be coupled to a capnography monitor. The capnography system 1 may further include an oxygen supply 3 configured to supply oxygen to the patient 2 via an oxygen tube 4. The cannula 10 may be coupled to the oxygen tube 4 and configured to deliver oxygen to the patient 2, for example, during or intermittent with receiving exhalation from the patient 2.

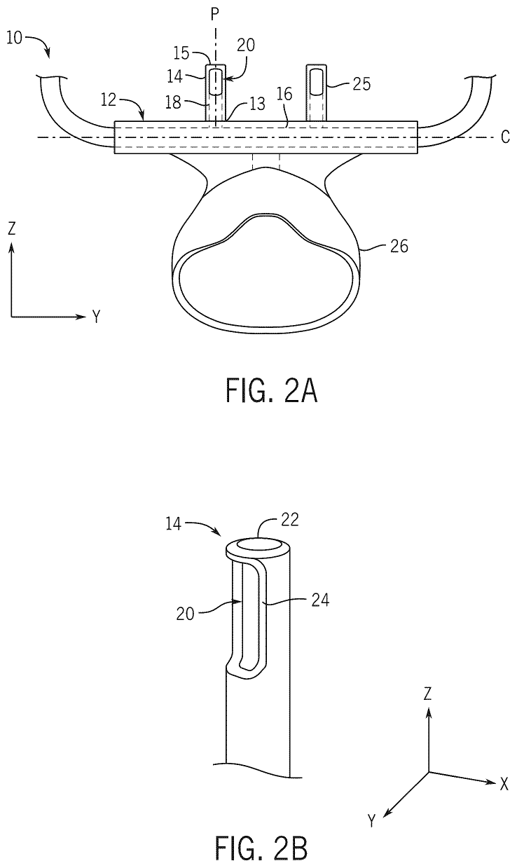

FIG. 2A is a rear perspective view of an embodiment of a cannula 10 including a cannula body 12 and at least one prong 14 extending from cannula body 12 and configured to receive a volume of exhalation from a patient. In some embodiments, the at least one prong 14 includes two prongs, and in other embodiments, the at least one prong 14 includes one prong or more than two prongs. Thus, while cannulas including two prongs are primarily described herein, in other embodiments, the cannulas described herein can include any suitable number of prongs.

The cannula body 12 defines an exhalation lumen 16 configured to be fluidically coupled to a capnography analysis module (for example, capnography analysis module 7 of FIG. 1). The exhalation lumen 16 may extend along a longitudinal cannula axis C (e.g., central exhalation lumen axis, shown to be extending in the x-axis direction, where orthogonal x-y-z axes are shown in the figures for ease of description only). Each prong 14 defines a prong lumen 18 fluidically coupled to the exhalation lumen 16. Each prong lumen 18 extends to a respective prong opening 20 (e.g., aperture) defined by the respective prong 14 and is configured to receive a volume of exhalation from the patient. At least a portion of prong opening 20 faces radially outward from a longitudinal prong axis P (e.g., central prong axis, shown to be extending in the z-axis direction) of prong 14. The longitudinal prong axis P extends from the cannula body 12 normal to (e.g., perpendicular to, orthogonal to) the cannula axis C, and a proximal end 13 of each respective prong 14 is closest to the cannula body 12 and a distal end 15 of each respective prong 14 is furthest from cannula body 12. The longitudinal axis P can be, for example, a central longitudinal axis of prong 14. Prong opening 20 may be configured to face the patient 2, for example, within a nostril of the patient 2 facing a rear wall of the nostril (e.g., in a posterior direction).

The cannula body 12 and the at least one prong 14 may be formed from any suitable material(s). For example, one or both of the cannula body 12 or the at least one prong 14 may include at least one polymer. The cannula body 12 and the at least one prong 14 may be similar or identical in a material or composition of construction, or may differ in one or more components of composition. For example, the cannula body 12 and the at least one prong 14 may include or be formed of the same polymer. The polymer may include any suitable compatible polymer, for example, a biocompatible or medical polymer. The polymer may be flexible to an extent, but sufficiently rigid to retain a shape of the cannula body 12 and the at least one prong 14. One or both of the cannula body 12 and the at least one prong 14 may be transparent, translucent, or opaque.

FIG. 2B is a partial perspective view of an embodiment of at least one prong 14 of FIG. 2A. In particular, the prong 14 extends to a prong end 22, which is also referred to herein as a prong distal end 22. In some embodiments, a geometric center of prong opening 20 is spaced from prong distal end 22 by a distance in a range from 5% to 100% of a length of the prong 14, the distance being measured in a direction along the longitudinal prong axis P. Additionally or alternatively, in some embodiments, a maximum width of prong opening 20, measured in a direction transverse to the longitudinal prong axis P of the prong 14, is in a range from 5% to 95% of a maximum diameter of the prong 14.

The prong 14 may be a first prong, and the cannula 10 may further include a second prong. For example, as shown in FIG. 2A, the cannula 10 may include a pair of prongs. The prongs may be spaced a distance apart from one another to be placeable within respective nostrils of a patient. The prong 14 and the second prong 25 may be sufficiently flexible to be accommodated within nostrils being separated by variable spacing in different individuals, for example, by bending or flexing. In some embodiments, different inter-prong spacings may be provided in different sizes or types, for example, for pediatric patients, adolescents, and adults having different nostril dimensions and spacing.

The cannula body 12 extends along the longitudinal cannula axis C (e.g., a central longitudinal axis), and prong 14 extends along the longitudinal prong axis P that is not parallel to the longitudinal cannula axis C. For example, the longitudinal prong axis P can be substantially normal to the longitudinal cannula axis C. For example, the longitudinal prong axis P may be exactly 90°, or 90°+5°, with respect to the longitudinal cannula axis C. In other embodiments, the longitudinal prong axis P may be inclined, for example, in a range from over 0°, for example, up to 90° with respect to the longitudinal cannula axis C. In embodiments in which a pair of prongs is present, the prongs may be inclined towards each other or away from each other.

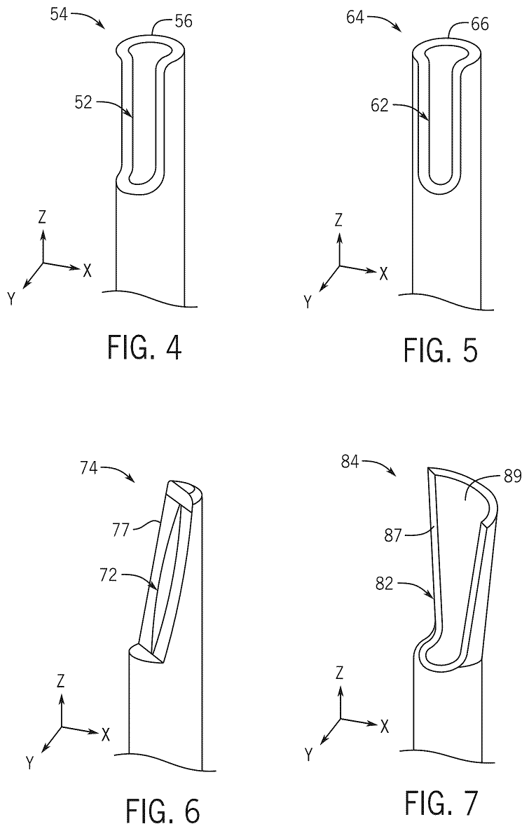

In some embodiments, as described with reference to FIGS. 4 to 7, a prong may extend to an open prong end. In other embodiments, at least one prong 14 extends from the cannula body 12 to a closed prong end. For example, as shown in FIG. 2B, the prong distal end 22 is closed. In these embodiments, the prong opening 20 is positioned between the closed prong distal end 22 and the cannula body 12. In some embodiments, the prong opening 20 may be closer to the prong distal end 22 than to the cannula body 12. For example, a center of the prong opening 20 may be spaced away from the cannula body 12 by more than 50% of a distance between the prong end 22 and the cannula body 12. In embodiments in which the prong distal end 22 is closed, an entire perimeter of the prong opening 20 is defined by a side surface (e.g., outer surface) of the prong 14, the side surface extending around the longitudinal prong axis P and defining the prong lumen 18.

Continuing with FIG. 2B, the prong opening 20 may define a pair of opposing prong edges 24. As shown in FIG. 2B, the prong edges 24 may be inset or recessed radially inward from an outer surface of the prong 14. In other embodiments, the prong edges 24 may not be inset or recessed. The opposing prong edges 24 may be substantially parallel to each other, or inclined relative to each other.

The prong opening 20 may have any suitable shape, for example, polygonal, rounded, ellipsoidal, circular, or curved. In some embodiments, the prong opening 20 is substantially a rounded rectangle. The prong opening 20 may include at least one connected or disconnected oval, circular, ellipsoidal, curved, or polygonal peripheral sections. For example, the sections may be connected or overlapping, or discrete. Accordingly, the prong opening 20 may include a single opening or a plurality of sub-openings.

In some embodiments, the at least one prong 14 is integral with the cannula body 12. For example, the prong 14 may be unitary with the cannula body 12 and extend continuously from the cannula body 12. In some embodiments, the at least one prong 14 may be discrete from the cannula body 12, but welded, adhered, overmolded, or otherwise coupled to the cannula body 12.

Prongs are configured to facilitate receiving exhalation from a patient, for example, into exhalation lumen 16, which in turn may be fluidically coupled to the capnography analysis module 7. Prongs that define openings only at distal ends may not adequately shield patient exhalation or carbon dioxide flow from oxygen being supplied to the patient, which may result in introduction and mixing of oxygen with exhalation. Such mixing may improperly indicate a reduced carbon dioxide content if such mixed exhalation is analyzed by a capnography analysis module. As such, at least one prong opening 20 may face radially outward from the longitudinal prong axis P to be substantially shielded or improve shielding of the at least one prong opening 20 from supplied oxygen. In particular, a remaining portion of prong 14 (e.g., at the distal prong end 22) may act as a shield or barrier against oxygen mixing into exhalation introduced into the prong opening 20. Thus, the prong opening 20 may promote improved accuracy of capnography measurements and data as compared to prong openings provided only at distal ends of the prongs.

With reference to FIG. 2A, in some embodiments, the cannula 10 further includes a mouthpiece 26 extending from the cannula body 12 and fluidically coupled to exhalation lumen 16. The mouthpiece 26 is configured to receive exhalation from a mouth of the patient. The mouthpiece 26 may have any suitable shape. For example, as shown in FIG. 2A, the mouthpiece 26 may be curved or contoured to facilitate placement and retention within the mouth. The mouthpiece 26 may be formed as a partially open pouch or envelope. The mouthpiece 26 may smoothly or continuously extend from the cannula body 12. The mouthpiece 26 may include a relatively flexible material, for example, a material that can conform to the inner contours of the mouth, but returning to an initial shape upon removal from the mouth. The mouthpiece 26 includes an opening coupled to the exhalation lumen 16. Thus, if a portion of exhalation is discharged by the patient through the mouth, the at least one prong 14 and the mouthpiece 26 may together substantially receive the entire exhalation generated by the patient.

In addition to receiving or collecting exhalation, cannulas according to the disclosure may further supply oxygen to the patient. Supplying oxygen through the cannula that also transports exhalation for capnography (capnography cannula) may reduce the number of devices in contact with the patient, for example, by avoiding the use of external or additional oxygen masks or oxygen supply devices. Supplying oxygen through the capnography cannula may also facilitate orienting oxygen flow towards a patient's nostrils, because placing the prong into a nostril may provide a reliable and consistent anchoring location. Thus, one or more oxygen delivery openings may be spaced or oriented relative to the prong to direct oxygen flow to the patient's nostril.

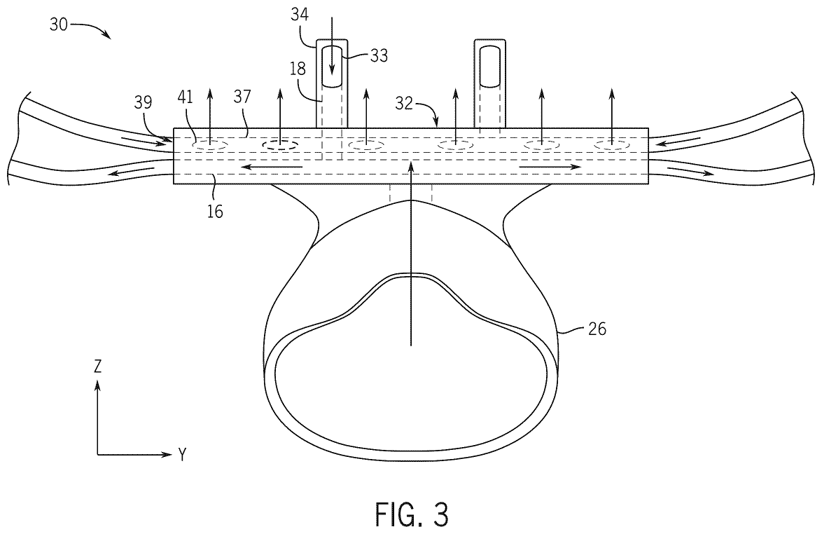

With the foregoing in mind, FIG. 3 is a rear perspective view of an embodiment of a cannula 30 including a cannula body 32, at least one prong 34, and at least one oxygen delivery opening 41 (e.g., aperture.) The cannula body 32 and the prong 34 may be substantially similar to the cannula body 12 and the prong 14 described with reference to FIGS. 2A and 2B. The cannula body 32 defines the exhalation lumen 16, and the prong 34 defines the prong lumen 18 fluidically coupled to the exhalation lumen 16. The cannula body 32 further defines an oxygen lumen 37 extending between an oxygen inlet 39 defined by the cannula body 32 and the at least one oxygen delivery opening 41 defined by the cannula body 32. The oxygen lumen 37 may be configured to transport oxygen to the patient via the at least one oxygen delivery opening 41. In some embodiments, the oxygen delivery opening 41 is positioned to face away from a prong opening 33 of the prong 34. Thus, while the prong opening 33 may face an inner wall (e.g., in a posterior direction) of a nostril of a patient, the at least one oxygen delivery opening 41 may face an outer wall (e.g., in an anterior direction) of the nostril, such that oxygen is still delivered to the nostril while being separated from exhalation received by the prong 34 via the prong opening 33. The cannula 30 may further include a mouthpiece 26 in some embodiments.

The at least one oxygen delivery opening 41 may have any suitable shape, for example, circular, ellipsoidal, polygonal, rounded polygonal, or curved, or a compound shape combining one or more elementary shapes. The cannula 30 may include a plurality of oxygen delivery openings including the oxygen delivery opening 41. The plurality of oxygen delivery openings may be positioned along a length of the cannula body 32. The plurality of oxygen delivery openings may be identical in shape and size, or may differ in one or both of shape or size. In addition, it should be appreciated that the cannula 30 may include any suitable number of oxygen delivery openings (e.g., 1, 2, 4, 5, 6, 10, etc.) to deliver a desired amount (e.g., volume) and/or at any desired rate of delivery (e.g., volume/min) to a patient.

While the prong distal end 22 is closed in the embodiments shown in FIGS. 2A and 2B, in other embodiments, the prong opening may extend to an open prong end, as described with reference to FIGS. 4 and 5. In embodiments in which the prong distal end 22 is open, at least part of a perimeter of the prong opening is defined by an end surface of the prong. However, in these embodiments, the prong opening is still defined, at least in part, by a side surface of a prong, e.g., such that the prong opening has a length that extends a long a longitudinal axis (e.g., a length) of the prong and such that at least part of (e.g., at least a portion of) the prong opening faces in a radially outward direction relative to a central longitudinal axis of the prong.

FIG. 4 is a partial view of an embodiment of a prong 54 defining a recessed prong opening 52 extending to an open prong end 56. The prong 54 is generally similar to the prong 14 other than differing in the presence of the open prong end 56 (also referred to as a prong distal end 56). The open prong end 56 may facilitate collecting exhalation in case of blockage of another portion of the prong opening 52, for example, by mucus or other fluids, or in case of a relative shift between the prong 54 and the patient's nostril.

FIG. 5 is a partial view of an embodiment of a prong 64 defining a prong opening 62 extending to an open prong end 66. The prong 64 is generally similar to the prong 14 other than differing in the presence of the open prong end 66. In addition, the prong opening 62 is generally similar to the prong opening 52 of FIG. 4 other than differing in that the prong opening 62 is not inset or recessed from a surface (e.g., outer surface) of the prong 64.

While at least a portion of a prong wall defining a prong opening may be substantially parallel to a longitudinal direction of a prong, in other embodiments, at least a portion of the prong wall defining a prong opening may be inclined relative to the prong.

FIG. 6 is a partial view of an embodiment of a prong 74 defining an inclined prong opening 72. For example, the prong 74 may define a prong axis extending along the z-axis direction, and the inclined prong opening 72 may be defined by a pair of opposing prong edges 77 (of prong 74) extending in a direction inclined relative to the prong axis. The opposing prong edges 77 may extend along straight lines, or may be curved, for example, convex or concave. The inclined prong opening 72 may be ellipsoidal, but otherwise may have any suitable shape described with reference to prong opening 20.

FIG. 7 is a partial view of an embodiment of a prong 84 including a pair of opposing prong edges 87 extending radially away from each other. Thus, the prong edges 87 may define a diverging prong opening 82 (e.g., diverging away from a central prong axis extending along the z-axis direction). Accordingly, a wall 89 of the prong 84 between the prong edges 87 may be shaped as a shield or a scoop. The wall 89 may be piecewise or continuously curved or flat. The wall 89 may define an increasing or decreasing wall diameter in a direction towards a distal prong end of the prong 84. The pair of opposing prong edges 87 may extend radially toward each other instead of extending away from each other in a direction towards the distal prong end of the prong 84.

While a tubular cannula body has been shown with reference to FIG. 2A, cannula bodies in cannulas according to the present disclosure may have any other suitable shaped, for example, rectanguloid, or rounded rectanguloid.

Additionally or alternatively, cannulas according to the present disclosure may further include oxygen delivery openings configured to facilitate separation of exhalation from oxygen. The one or more oxygen delivery openings may be sized or shaped to provide a more uniform or symmetric oxygen flow, compared to other shapes of oxygen delivery openings. For example, an oxygen delivery opening may be elongated, and have a major dimension that is longer than a minor dimension. In some embodiments, the one or more oxygen delivery openings are positioned relative to the prongs to separate or space oxygen outflow delivered through oxygen delivery openings from exhalation inflow received into the prongs openings.

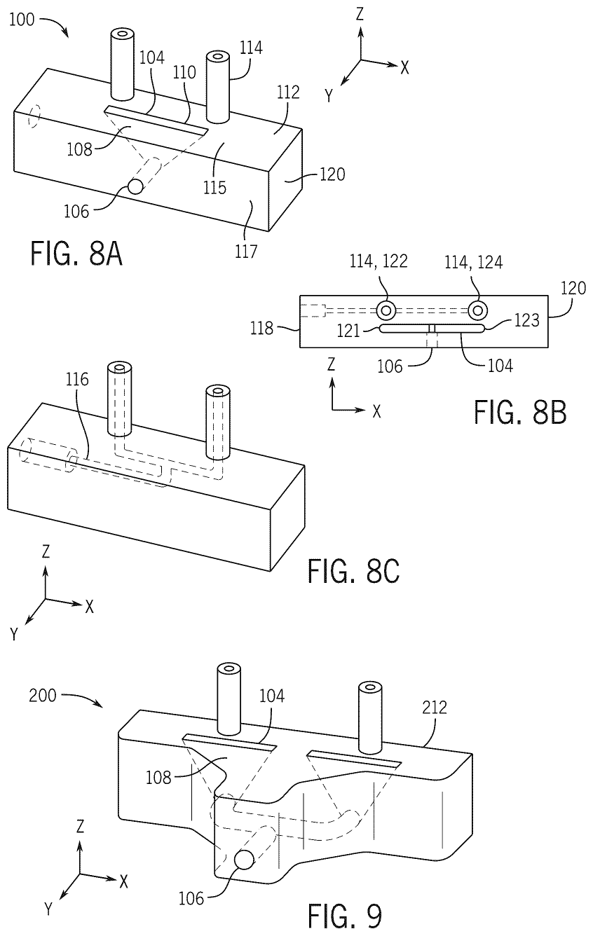

FIG. 8A is a perspective view of an embodiment of a cannula 100 including a cannula body 112 and at least one elongated oxygen delivery opening 104. FIG. 8B is a top perspective view of cannula 100 of FIG. 8A. The cannula body 112 may be similar in material or construction to the cannula body 12 described with reference to FIG. 2A. For example, the cannula body 112 may include a polymer, and be tubular in form. However, as shown in FIG. 8A, cannula body 112 may be rectanguloid.

The cannula body 112 defines an exhalation lumen 116 (shown in the perspective view of FIG. 8C) configured to receive a volume of exhalation from a patient. The exhalation lumen 116 may transport the volume of exhalation toward a capnography analysis module.

The cannula body 112 further defines the elongated oxygen delivery opening 104 configured to deliver oxygen to the patient, for example, to nostrils of the patient. The elongated oxygen delivery opening 104 extends along a surface 115 (e.g., face, side, wall) of the cannula body 112 and is fluidically coupled to an oxygen inlet 106 by a distribution section 108. The surface 115 of the cannula body 112 may be the same surface 115 from which one or more prongs 114 extend. The oxygen inlet 106 is defined by the cannula body 112, for example, at an additional surface 117 of cannula body 112. The distribution section 108 is defined by the cannula body 112 and has a major cross-sectional area greater than a major cross-sectional area of the oxygen inlet 106.

In some embodiments, the distribution section 108 is configured to expand or increase a cross-sectional area between the oxygen inlet 106 and the elongated oxygen delivery opening 104, to distribute oxygen supply from the oxygen inlet 106 to the elongated oxygen delivery opening 104. Thus, the distribution section 108 may permit the use of a smaller or narrower oxygen inlet 106 than a maximum width of the elongated oxygen delivery opening 104, or otherwise a smaller cross-sectional area for the oxygen inlet 106 than for the elongated oxygen delivery opening 104. Oxygen from an oxygen supply supplied to the oxygen inlet 106 may to flow along a path guided by the distribution section 108, which may create a three-dimensional distribution of oxygen flow to both nostrils, and reduce the velocity of the supplied oxygen emerging (e.g., exiting, expelled) from elongated oxygen delivery opening 104.

The distribution section 108 may also facilitate uniform flow of oxygen, for example, by reducing turbulence between the oxygen inlet 106 and the elongated oxygen delivery opening 104. The distribution section 108 may be generally triangular, as shown in FIG. 8A, but may have any other suitable shape. The distribution section 108 may include one or more sections that do not change in cross-sectional area, but collectively may provide an increased cross-sectional area relative to the oxygen inlet 106.

The oxygen inlet 106 may have any suitable shape, for example, circular, ellipsoidal, square, rectangular, triangular, polygonal, or rounded polygonal.

The elongated oxygen delivery opening 104 may define an opening periphery 110 (e.g., edge, perimeter) along the surface 115 of cannula body 112. The opening periphery 110 may be one of rectangular, rounded rectangular, n-sided polygonal, piecewise linear, piecewise curved, or curved. In some embodiments, the opening periphery 110 is a rectangle, a triangle, a rhombus, a kite, a trapezium, or a parallelogram. At least one vertex of the opening periphery 110 may be sharp, angled, curved, or rounded.

The cannula body 112 extends longitudinally between a first cannula end 118 and a second cannula end 120. The elongated oxygen delivery opening 104 (e.g., a length of the elongated oxygen delivery opening 104) may extend in a direction generally along a direction from the first cannula end 118 to the second cannula end 120. For example, the elongated oxygen delivery opening 104 may be longer along one axis (for example, the x-axis) than along another axis (for example, the y-axis). In other embodiments, the elongated oxygen delivery opening 104 may extend transverse to a direction between first cannula end 118 and a second cannula end 120. For example, the elongated oxygen delivery opening 104 may be longer along the y-axis than along the x-axis.

In some embodiments, the oxygen inlet 106 is at a location spaced from the first cannula end 118 and the second cannula end 120 (e.g., longitudinally between the ends 118, 120.) In some embodiments the oxygen inlet 106 may be aligned with a longitudinal center of the distribution section 108 in a direction along the elongated oxygen delivery opening 104.

The cannula 100 may further include at least one prong 114 extending from the surface 115 of cannula body 112. The prong 114 may be tubular with an opening at the end, or be similar to prongs 14, 34, 54, 64, 74, or 84 described elsewhere in the present disclosure.

The at least one prong 114 may include a pair of prongs positioned along a line parallel to at least one elongated delivery opening 104. The pair of prongs may be aligned with the elongated oxygen delivery opening 104. In some embodiments, the pair of prongs may flank the elongated oxygen delivery opening 104, for example, being spaced from and adjacent opposing ends of the elongated oxygen delivery opening 104. The pair of prongs may be in the same line along which the elongated oxygen delivery opening 104 extends, or may extend along a parallel line. The elongated oxygen delivery opening 104 may extend from a first opening end 121 spaced from a first prong 122 of the pair of prongs 114 to a second opening end 123 spaced from a second prong 124 of the pair of prongs 114.

In other embodiments, a cannula can include more than one oxygen delivery opening, such as two, three, or more than three oxygen delivery openings. FIG. 9 is a perspective view of an embodiment of a cannula 200 including a pair of elongated oxygen delivery openings 104 fluidically coupled to oxygen inlet 106. Each elongated oxygen delivery opening 104 extends from a corresponding distribution section 108 fluidically coupled to the oxygen inlet 106. The elongated oxygen delivery openings 104 or respective distribution sections 108 may be identical in size or shape, or may differ in on or both of size or shape. While two elongated oxygen delivery openings 104 or respective distribution sections 108 are shown in FIG. 9, more than two elongated oxygen delivery openings 104 or respective distribution sections 108 may be provided. The cannula 200 may include a cannula body 212 generally similar to cannula body 112, but differ in exterior contour and shape to accommodate the additional number(s) of elongated oxygen delivery openings 104 or respective distribution sections 108. For example, a center section (e.g., portion) of the cannula body 212 may protrude toward the oxygen inlet 106.

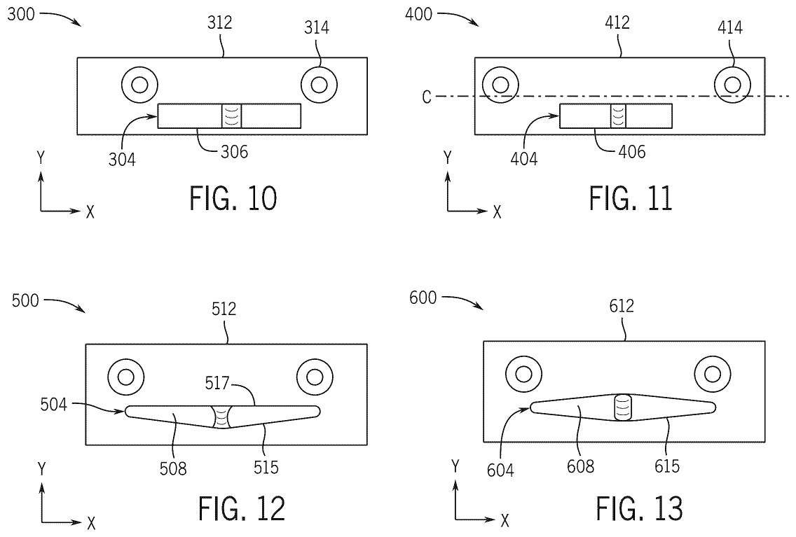

The elongated oxygen delivery openings or distribution sections having different shapes or contours may be useful in cannulas according to the present disclosure, as described with reference to FIGS. 10 to 28.

FIG. 10 is a top perspective view of an embodiment of a cannula 300 including a cannula body 312 defining an elongated oxygen delivery opening 304 having a rectangular periphery 306 extending between a pair of prongs 314. For example, the elongated oxygen delivery opening 304 extends entirely between prongs 314 such that no portion of the elongated oxygen delivery opening 304 overlaps with either prong of the pair of prongs 314, or otherwise extends beyond prongs 314.

FIG. 11 is a top perspective view of an embodiment of a cannula 400 including a cannula body 412 defining an elongated oxygen delivery opening 404 between and spaced from a pair of prongs 414. Compared to elongated oxygen delivery opening 304 of cannula 300, the elongated oxygen delivery opening 404 may be shorter in length, the length being measured along a longitudinal cannula axis C of cannula body 412. Alternatively, or additionally, the spacing between prongs 414 may be greater than that between prongs 314 of cannula 300. Such different relative spacing may be useful to conform to patients having different oral or facial anatomy.

FIG. 12 is a top perspective view of an embodiment of a cannula 500 including a cannula body 512 defining an elongated oxygen delivery opening 504 extending from a distribution section 508 including an angled wall 515 and a flat wall 517. For example, The distribution section 508 may include the angled wall 515 opposing the flat wall 517. The angled wall 515 may include one or more wall portions that extend at an angle with respect to each other, such that the one or more wall portion meet at a point or vertex. The angled wall 515 and the flat wall 517 may be connected by rounded ends. A shape of the elongated oxygen delivery opening 504 may be substantially triangular.

FIG. 13 is a top perspective view of an embodiment of a cannula 600 including a cannula body 612 defining an elongated oxygen delivery opening 604 extending from a distribution section 608 including a pair of opposed angled walls 615. The angled walls 615 may define sharp or rounded ends or vertices. A shape of the elongated oxygen delivery opening 504 may be substantially rhombus or diamond.

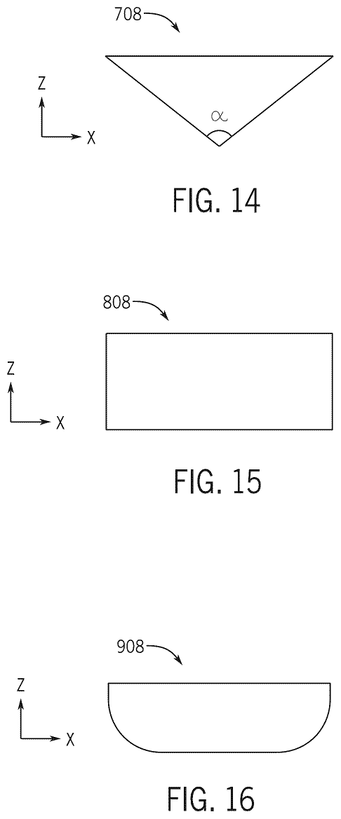

FIG. 14 is a front schematic view of a contour of a distribution section 708 including a triangular wall. The triangular wall may subtend any suitable angle α, which may be acute, 90°, or obtuse.

FIG. 15 is a front schematic view of a contour of a distribution section 808 having a rectangular wall.

FIG. 16 is a front schematic view of a contour of a distribution section 908 having a boat-shaped wall. For example, the wall may have a pair of curvilinear, curved, or linear edges extending between a wide first edge and a narrow second edge. In some embodiments, the wall may have a rounded bottom.

The contours of FIGS. 14 to 16 may be used with the wall configurations, peripheries, or inter-prong or inter-prong-delivery opening spacings or orientations described with respect to any cannula according to the present disclosure, for example, cannulas of any of FIGS. 2A to 13.

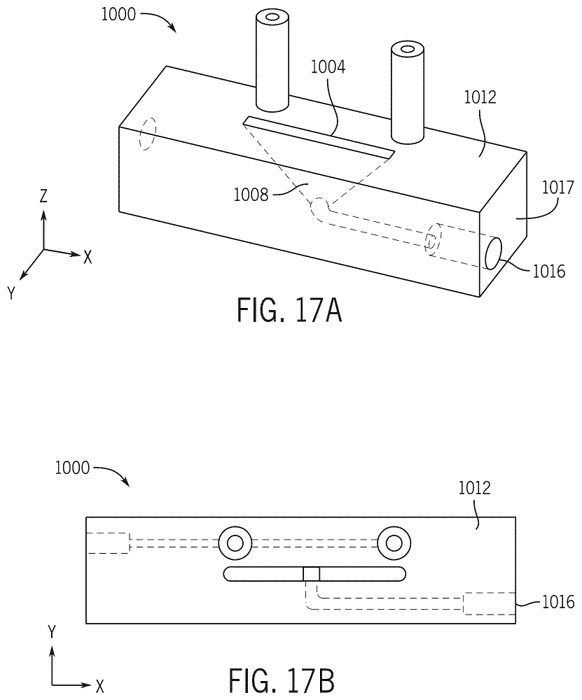

While an oxygen inlet is positioned at or adjacent a center of a cannula body or a center of a distribution section in some embodiments, in other embodiments, an oxygen inlet is located along or at an end, a side, or a corner of a cannula body, as described with reference to FIGS. 17A to 19B. For example, positioning an oxygen inlet along or at an end, a side, or a corner of a cannula body may avoid placing oxygen tubing or coupling mechanisms facing the nose of the patient, and reduce a bulk of the cannula extending toward the nose. Such positioning may also facilitate checking and adjustment of couplings between oxygen tubing and the cannula by a clinician, for example, without requiring moving cannula away from the patient's nose.

Moving the oxygen inlet to the side may introduce some asymmetry to oxygen flow received from the inlet. For example, a portion of an oxygen lumen closer to the inlet may receive oxygen at a relatively higher flow rate or pressure compared to another portion of the oxygen lumen farther away from the inlet. In some embodiments, one or both of elongated oxygen delivery openings and distribution sections according to the present disclosure are configured to promote uniform oxygen supply even if an oxygen inlet is positioned at a side, and end, or a corner of a cannula, for example, by distributing or dispersing oxygen across a varying or increasing cross-section from the oxygen inlet to the oxygen openings. For example, a size, a shape, a position along the cannula body of one or more openings used to deliver oxygen to a patient may be selected to route the oxygen flow to the patient in a substantially symmetrical manner (e.g., symmetrical relative to prongs of the cannula, nostrils of a patient wearing the cannula, or other reference point or nearly symmetrical to the extent permitted by manufacturing tolerances). In some embodiments, the one or more openings may be substantially symmetrical relative to two prongs of the cannula body, and thus, relative to two nostrils of the patient. The symmetrical oxygen flow may direct a substantially similar volumetric flow rate and pressure of oxygen to each nostril. In some embodiments, the one or more openings may be configured to modify oxygen flow velocity, such that oxygen emerging from the openings has a velocity that reduces mixing with exhaled breath. For example, a relatively slower oxygen velocity may result in relatively lower mixing of the oxygen with exhaled breath, thus maintaining or improving the accuracy of capnography relative to cannulas through which oxygen is delivered at relatively higher velocities, thus maintaining accuracy at higher overall flow rates.

FIG. 17A is a perspective view of an embodiment of a cannula 1000 including a cannula body 1012 defining an elongated oxygen delivery opening 1004 extending from a distribution section 1008 coupled to a side oxygen inlet 1016. FIG. 17B is a top perspective view of cannula 1000 of FIG. 17A. Providing the oxygen inlet 1016 at a side surface or an end surface 1017 of cannula body 1012 may promote compactness in a region adjacent the patient's nose or nostrils, and may reduce a bulk of the cannula near the patient's nose.

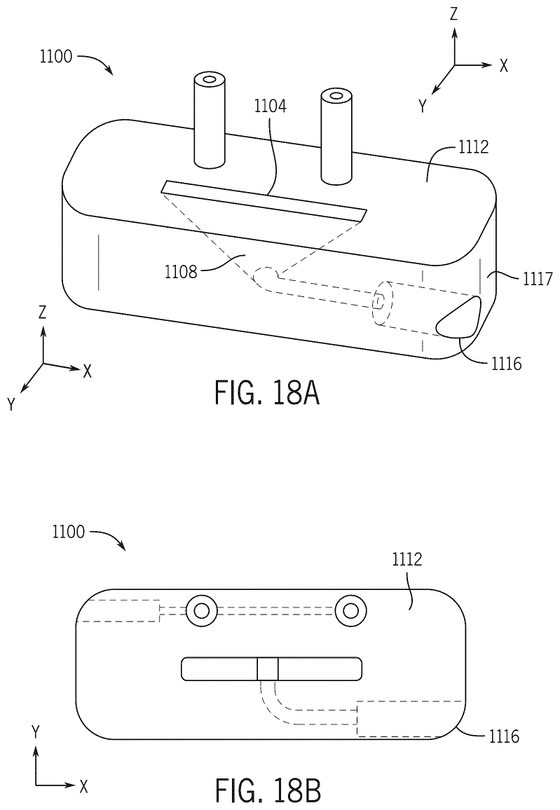

FIG. 18A is a perspective view of an embodiment of a cannula 1100 including a rounded cannula body 1112 defining an elongated oxygen delivery opening 1104 extending from a distribution section 1108 coupled to a side oxygen inlet 1116. FIG. 18B is a top perspective view of cannula 1110 of FIG. 18A. As seen in FIGS. 18A and 18B, the oxygen inlet 1116 may be positioned at a rounded corner or a rounded end 1117 of the cannula body 1112. Providing the oxygen inlet 1116 at a corner 1117 of cannula body 1112 may promote compactness in a region adjacent the patient's nose or nostrils, and may reduce a bulk of the cannula near the patient's nose.

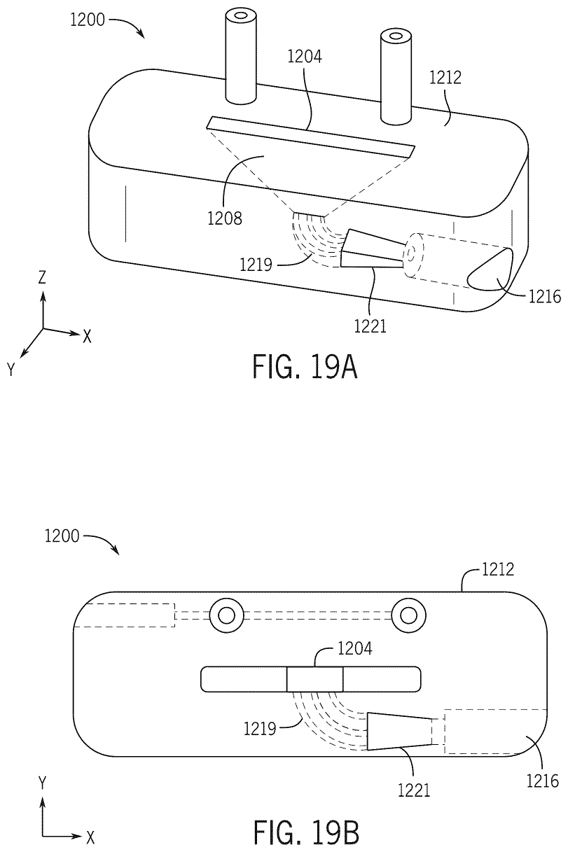

FIG. 19A is a perspective view of an embodiment of a cannula 1200 including a cannula body 1212 defining an elongated oxygen delivery opening 1204 extending from a distribution section 1208 coupled to a side oxygen inlet 1216 by a plurality of inlet lumens 1219. FIG. 19B is a top perspective view of the cannula 1200 of FIG. 19A. The cannula 1200 may further include a diverging conduit 1221 between the oxygen inlet 1216 and the plurality of inlet lumens 1219. The diverging conduit 1221 and the plurality of inlet lumens 1219 may distribute and diverge oxygen supplied from the oxygen inlet 1216 to the elongated oxygen delivery opening 1204, which may reduce turbulence, and otherwise facilitate a uniform and distributed flow of oxygen to one or both nostrils of the patient.

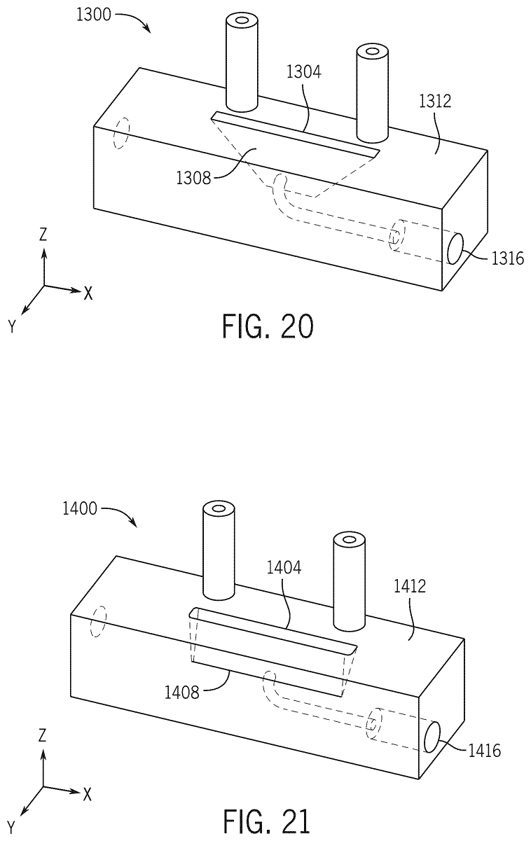

FIG. 20 is a perspective view of an embodiment of a cannula 1300 including a cannula body 1312 defining an elongated oxygen delivery opening 1304 extending from a distribution section 1308 coupled to a side oxygen inlet 1316 at a location laterally offset from a center of distribution section 1308. For example, the oxygen inlet 1316 may be laterally offset from the center of distribution section 1308 in a direction along a length of the elongated oxygen delivery opening 1304.

FIG. 21 is a perspective view of an embodiment of a cannula 1400 including a cannula body 1412 defining an elongated oxygen delivery opening 1404 extending from a wedge-shaped distribution section 1408.

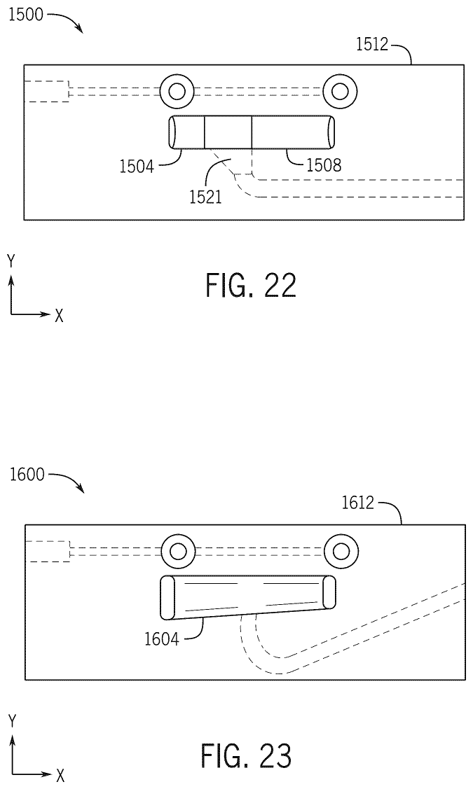

FIG. 22 is a perspective view of an embodiment of a cannula 1500 including a cannula body 1512 defining an elongated oxygen delivery opening 1504 fluidically coupled to an oxygen inlet 1516 by a diverging asymmetric supply conduit 1521.

FIG. 23 is a perspective view of an embodiment of a cannula 1600 including a cannula body 1612 defining an elongated asymmetric oxygen delivery opening 1604. The elongated asymmetric oxygen delivery opening 1604 may be asymmetric in one or more of lengths or orientations of edges or peripheral sections. For example, the elongated oxygen delivery opening 1604 may define a pair of opposed elongated edges having different lengths. Additionally, or alternatively, the edges may be non-parallel in an asymmetrical manner.

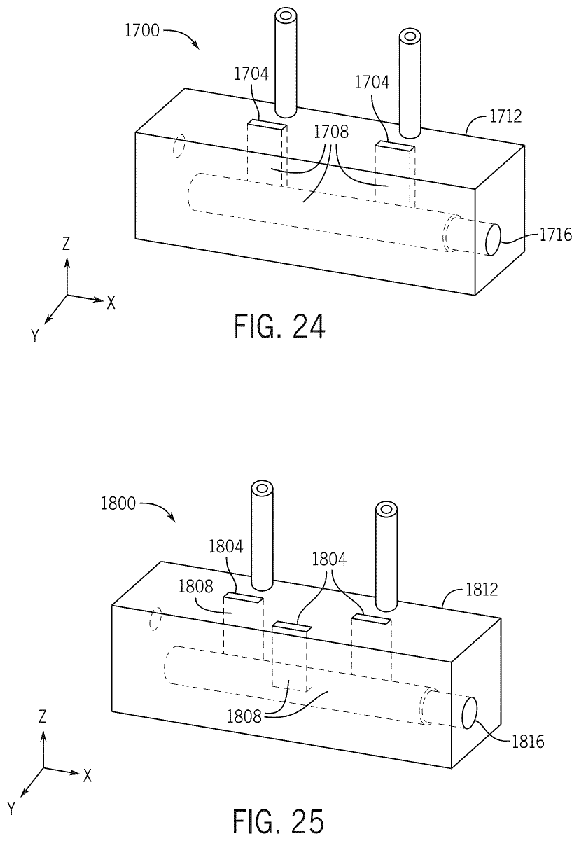

FIG. 24 is a perspective view of an embodiment of a cannula 1700 including a cannula body 1712 defining one or more distribution sections 1708 extending to a plurality of elongated oxygen delivery openings 1704 extending along a longitudinal axis of cannula body 1712. For example, the cannula body 1712 may extend along a longitudinal axis, and the elongated oxygen delivery opening 1704 may include at least two elongated oxygen delivery openings extending along the longitudinal axis. The distribution section 1708 may include a plurality of oxygen conduits. For example, each oxygen conduit of the plurality of oxygen conduits may fluidically couple a respective elongated oxygen delivery opening 1704 to an oxygen inlet 1716.

FIG. 25 is a perspective view of an embodiment of a cannula 1800 including a cannula body 1812 defining one or more distribution sections 1808 extending to a trio of elongated oxygen delivery openings 1804 extending along a longitudinal axis of cannula body 1812. The distribution section 1808 is fluidically coupled to an oxygen inlet 1816.

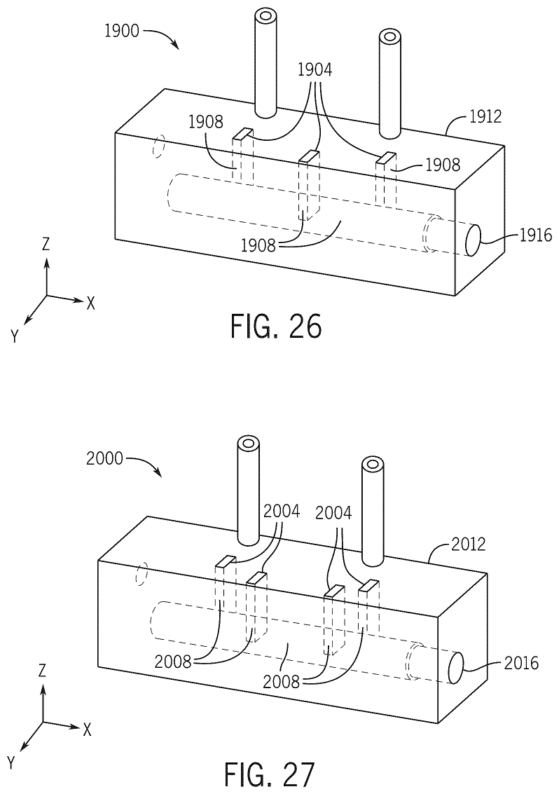

FIG. 26 is a perspective view of an embodiment of a cannula 1900 including a cannula body 1912 defining one or more distribution sections 1908 extending to a plurality of elongated oxygen delivery openings 1904 that extend transverse to a longitudinal axis of the cannula body 1912. The distribution section 1908 is fluidically coupled to an oxygen inlet 1916. For example, the distribution section 1908 may include a plurality of oxygen conduits. Each oxygen conduit of the plurality of oxygen conduits may fluidically couple a respective elongated oxygen delivery opening 1904 to an oxygen inlet 1916.

FIG. 27 is a perspective view of an embodiment of a cannula 2000 including a cannula body 2012 defining one or more distribution sections 2008 extending to four elongated oxygen delivery openings 2004 that extend transverse to a longitudinal axis of cannula body 2012. The distribution section 2008 is fluidically coupled to an oxygen inlet 2016.

As discussed herein, at least one oxygen delivery opening may have any suitable shape, for example, circular, ellipsoidal, polygonal, rounded polygonal, or curved, or a compound shape combining one or more elementary shapes. The cannula may include a plurality of oxygen delivery openings positioned along a length of the cannula body. The plurality of oxygen delivery openings may be identical in shape and size, or may differ in one or both of shape or size.

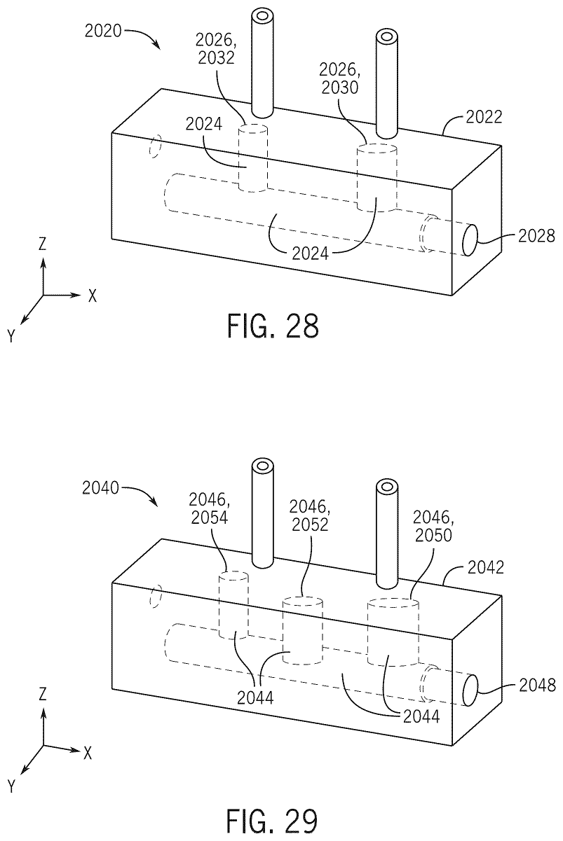

FIG. 28 is a perspective view of an embodiment of a cannula 2020 including a cannula body 2022 defining one or more distribution sections 2024 extending to and fluidly coupled to a plurality of circular or ovoid/elliptical oxygen delivery openings 2026 extending along a longitudinal axis of cannula body 2022. For example, the cannula body 2022 may extend along a longitudinal axis, and the circular oxygen delivery opening 2026 may include at least two circular oxygen delivery openings extending along the longitudinal axis. The distribution section 2024 may include a plurality of oxygen conduits. For example, each oxygen conduit of the plurality of oxygen conduits may fluidically couple a respective circular oxygen delivery opening 2026 to an oxygen inlet 2028. In particular, one or more of the oxygen delivery openings 2026 may be substantially circular in shape. In addition, to facilitate equal distribution of oxygen supply through each of the plurality of circular oxygen delivery openings 2026, a size or diameter of each circular oxygen delivery opening 2026 may vary. The diameter of the oxygen delivery openings 2026 may be relatively constant along the length of the passage of the distribution section 2024 formed in the cannula 2020 and terminating in the openings 2026. For example, a circular oxygen delivery opening 2026 positioned proximal to the oxygen inlet 2028 may include a diameter that is greater than a diameter of a circular oxygen delivery opening 2026 that is positioned distal to the oxygen inlet 2028. In some embodiments, each diameter or size of a respective (e.g., sequential, successive) circular oxygen delivery opening 2026 may decrease as a respective distance from the oxygen inlet 2028 increases. As in FIG. 28, a first circular oxygen delivery opening 2030 is positioned along the longitudinal axis at a first distance from the oxygen inlet 2028 that is less than a distance at which a second circular oxygen delivery opening 2032 is positioned. In addition, the first oxygen delivery opening 2030 has a diameter that is greater than a diameter of the second circular oxygen delivery opening 2032. In an embodiment, the diameter of the first oxygen delivery opening 2030 is at least 10%, at least 25%, or at least 100% greater than the diameter of the second circular oxygen delivery opening 2032.

FIG. 29 is a perspective view of an embodiment of a cannula 2040 including a cannula body 2042 defining one or more distribution sections 2044 extending to and fluidly coupled to a trio of circular oxygen delivery openings 2046 extending along or with respect to a longitudinal axis of cannula body 2042. The distribution section 2044 is fluidically coupled to an oxygen inlet 2048. As in FIG. 29, a first circular oxygen delivery openings 2050 is positioned with respect to the longitudinal axis at a distance from the oxygen inlet 2048 that is less than each respective distance at which a second and a third circular oxygen delivery openings 2052, 2054 are positioned. In addition, the second circular oxygen delivery opening 2052 is positioned with respect to the longitudinal axis at a distance that is less than the respective distance at which the third circular oxygen delivery opening 2054 is positioned. Furthermore, the first circular oxygen delivery openings 2050 includes a diameter that is greater than each respective diameter of the second and third circular oxygen delivery openings 2052, 2054, while the second circular oxygen delivery openings 2052 includes a diameter that is greater than a diameter of the third circular oxygen delivery openings 2054. Each of the one or more circular oxygen delivery openings 2046 may be aligned with or extend along a central longitudinal axis of the cannula body 2042. In some embodiments, one or more of the circular oxygen delivery openings 2046 may be offset from the central longitudinal axis.

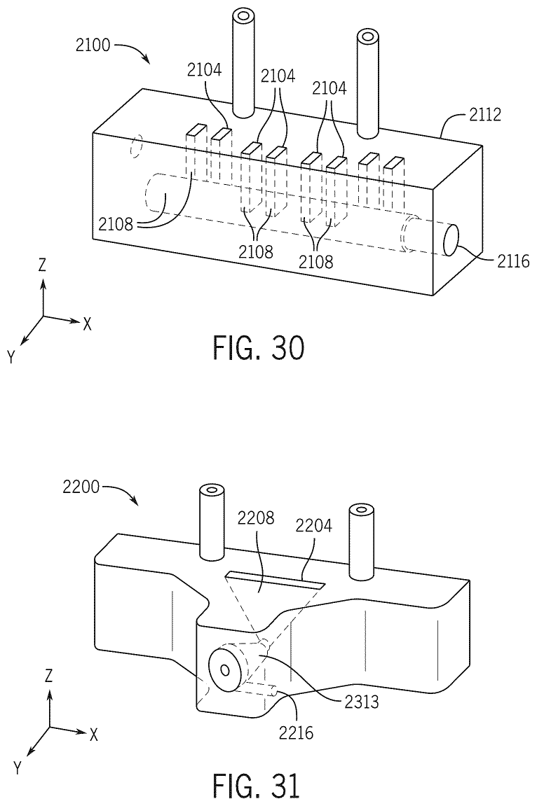

FIG. 30 is a perspective view of an embodiment of a cannula 2100 including a cannula body 2112 defining one or more distribution sections 2108 extending to and fluidly coupled to a plurality of pairs of elongated oxygen delivery openings 2104 extending transverse to a longitudinal axis of cannula body 2112. The distribution section 2108 may include a plurality of oxygen conduits. For example, each oxygen conduit of the plurality of oxygen conduits may fluidically couple a respective elongated oxygen delivery opening 2104 to an oxygen inlet 2116. The plurality of oxygen conduits may provide alternative flow paths in case of blockage of one or more conduits, and may facilitate distributing an incoming oxygen stream into multiple uniform sub-streams having substantially similar flow rates to be delivered through respective openings of pairs of elongated oxygen delivery openings 2104. In addition, the oxygen flow is divided to many outlet flows, reducing the gas velocity and promoting the accuracy of the sampling, since the exhalation of the patient will overcome the incoming oxygen flow more easily compared to an undistributed or undivided oxygen flow. Thus, dilution of exhalation may be reduced or prevented, resulting in more accurate capnography readings compared to readings in the presence of an undivided oxygen flow, while maintaining inlet position on one side. Such a configuration may promote a uniform, symmetric and low velocity oxygen supply to both nostrils while allowing oxygen supply inlet 2116 to be asymmetrically positioned on one side of cannula 2100.

FIG. 31 is a perspective view of an embodiment of a cannula 2200 including a cannula body 2212 defining an elongated oxygen delivery opening 2204 extending from a distribution section 2208 coupled to an oxygen vortex 2213. The oxygen vortex 2213 may fluidically couple an oxygen inlet 2216 to the distribution section 2208. The oxygen vortex 2213 may create a vortex flow, distributing the flow of oxygen in a time-dependent manner, in turn providing a symmetrical oxygen flow over time along the elongated oxygen delivery opening 2204. Further, frequency of oxygen flow can be adjusted with internal volume geometry of the oxygen vortex 2213.

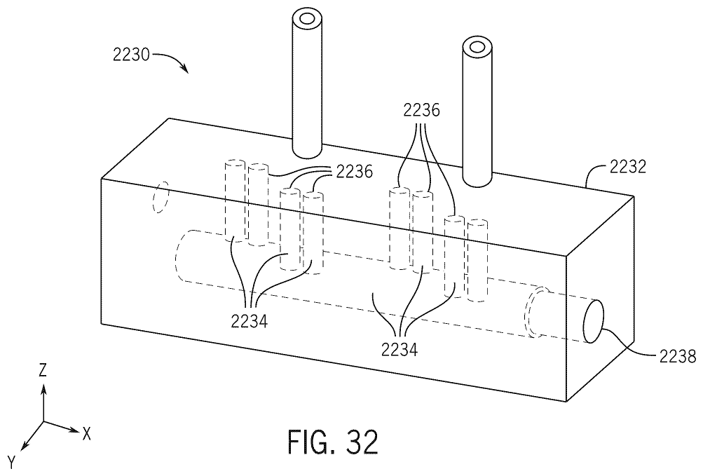

FIG. 32 is a perspective view of an embodiment of a cannula 2230 including a cannula body 2232 defining one or more distribution sections 2234 extending to and fluidly coupled to a plurality of pairs of circular oxygen delivery openings 2236 extending transverse to a longitudinal axis of cannula body 2232. The distribution section 2234 may include a plurality of oxygen conduits. For example, each oxygen conduit of the plurality of oxygen conduits may fluidically couple a respective elongated oxygen delivery opening 2236 to an oxygen inlet 2238. The plurality of oxygen conduits may provide alternative flow paths in case of blockage of one or more conduits, and may facilitate distributing an incoming oxygen stream into multiple uniform sub-streams having substantially similar flow rates to be delivered through respective openings of pairs of circular oxygen delivery openings 2236. In addition, the oxygen flow is divided to many outlet flows, reducing the gas velocity and promoting the accuracy of the sampling, since the exhalation of the patient will overcome the incoming oxygen flow more easily compared to an undistributed or undivided oxygen flow. Thus, dilution of exhalation may be reduced or prevented, resulting in more accurate capnography readings compared to readings in the presence of an undivided oxygen flow, while maintaining inlet position on one side. In some embodiments, one or more paired diameters of one or more of the pairs of circular oxygen delivery opening 2236 may vary. For example, a pair of circular oxygen delivery openings 2236 position proximal to the oxygen inlet 2238 may include a pair of diameters that is greater than a pair of diameters of a pair of circular oxygen delivery openings 2236 positioned distal to the oxygen inlet 2238. In other embodiments, each circular oxygen delivery opening (e.g., or each pair of circular oxygen delivery openings) may have a same diameter (e.g., same pair of diameters). Such a configuration may promote a uniform, symmetric and low velocity oxygen supply to both nostrils while allowing oxygen supply inlet 2238 to be asymmetrically positioned on one side of cannula 2230.

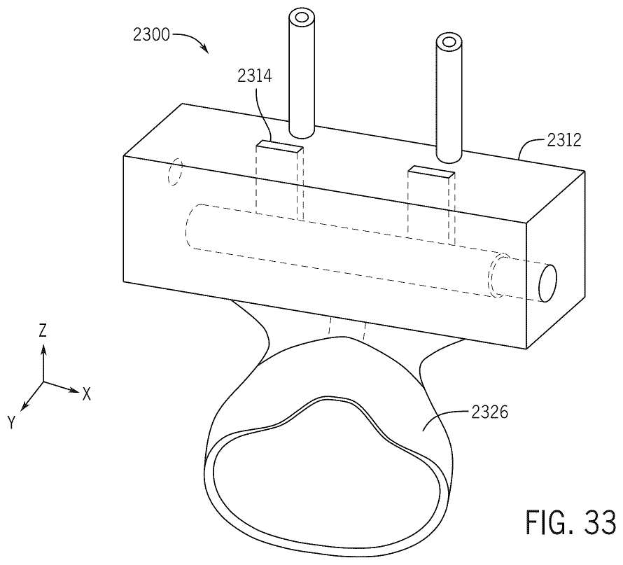

FIG. 33 is a perspective view of an embodiment of a cannula 2300 including a cannula body 2312 defining one or more elongated oxygen delivery openings 2314 and including a mouthpiece 2326 configured to receive exhalation from a patient. For example, the mouthpiece 2326 may extend from cannula body 2312 and be fluidically coupled to an exhalation lumen (not shown in FIG. 35). While cannula body 2312 in FIG. 35 is generally similar to cannula body 1712 described with reference to FIG. 24, cannula 2300 may include any suitable cannula body according to the present disclosure coupled to the mouthpiece 2326. The mouthpiece 2326 may be generally similar to the mouthpiece 26 described with reference to FIG. 3.

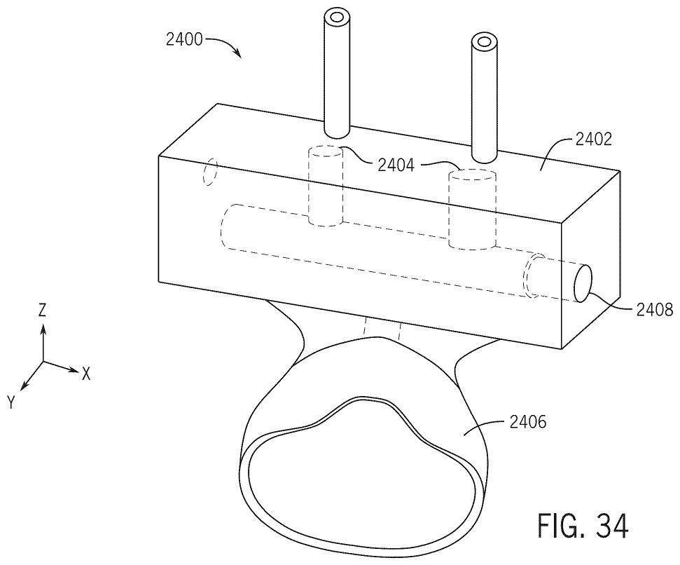

FIG. 34 is a perspective view of an embodiment of a cannula 2400 including a cannula body 2402 defining one or more circular oxygen delivery openings 2404 and including a mouthpiece 2406 configured to receive exhalation from a patient. For example, the mouthpiece 2406 may extend from cannula body 2312 and be fluidically coupled to an exhalation lumen (not shown in FIG. 34). While cannula body 2402 in FIG. 34 is generally similar to cannula body 1712 described with reference to FIG. 24, cannula 2400 may include any suitable cannula body according to the present disclosure coupled to the mouthpiece 2406. The mouthpiece 2406 may be generally similar to the mouthpiece 26 described with reference to FIG. 3. Similarly as discussed herein, a circular oxygen delivery opening 2404 positioned proximal to an oxygen inlet 2408 of the cannula 2400 may include a diameter that is greater than a diameter of a circular oxygen delivery opening 2404 that is positioned distal to the oxygen inlet 2408. In some embodiments, each diameter or size of a respective (e.g., sequential, successive) circular oxygen delivery opening 2026 may decrease as a respective distance from the oxygen inlet 2408 increases.

A system may include a capnography analysis module and a cannula according to the present disclosure fluidically coupled to the capnography analysis module to deliver the volume of exhalation to the capnography analysis module. The system may further include an oxygen supply, and the cannula may be further fluidically coupled to the oxygen supply to deliver oxygen to the patient.

The following enumerated examples are described herein.

Example 1: A cannula including: a cannula body defining: an exhalation lumen configured to receive a volume of exhalation from a patient; an oxygen inlet at a surface of the cannula body; at least one elongated oxygen delivery opening configured to deliver oxygen to nostrils of the patient, the at least one elongated oxygen delivery opening extending along a surface of the cannula body and fluidically coupled to the oxygen inlet by a distribution section defined by the cannula body, the distribution section having a major cross-sectional area greater than a major cross-sectional area of the oxygen inlet.

Example 2: The cannula of example 1, where the at least one elongated oxygen delivery opening includes a pair of elongated oxygen delivery openings.

Example 3: The cannula of examples 1 or 2, where the at least one elongated oxygen delivery opening defines an opening periphery along the surface, and where the opening periphery is one of rectangular, rounded rectangular, n-sided polygonal, piecewise linear, piecewise curved, or curved.

Example 4: The cannula of any of examples 1 to 3, further including at least one prong extending from the surface of the cannula body.

Example 5: The cannula of example 4, where the at least one prong includes a pair of prongs positioned along a line parallel to the at least one elongated delivery opening, and where the prongs flank the elongated oxygen delivery opening.

Example 6: The cannula of example 5, where the at least one elongated oxygen delivery opening extends from a first opening end spaced from a first prong of the pair of prongs to a second opening end spaced from a second prong of the pair of prongs.

Example 7: The cannula of any of examples 1 to 6, where the distribution section includes a pair of opposed triangular walls.

Example 8: The cannula of any of examples 1 to 6, where the distribution section includes a pair of opposed rectangular walls.

Example 9: The cannula of any of examples 1 to 8, where the distribution section includes a pair of opposed boat-shaped walls.

Example 10: The cannula of any of examples 1 to 9, where the distribution section includes an angled wall opposing a flat wall.

Example 11: The cannula of any of examples 1 to 10, where the distribution section includes a pair of opposed angled walls.

Example 12: The cannula of any of examples 1 to 11, where the cannula body extends longitudinally between a first cannula body end and a second cannula body end, and where the oxygen inlet is at a location spaced from the first cannula end and the second cannula end.

Example 13: The cannula of any of examples 1 to 11, where the oxygen inlet is located along an end or along a corner of the cannula body.

Example 14: The cannula of any of examples 1 to 13, where the oxygen inlet is aligned with a center of the distribution section in a direction along the at least one elongated oxygen delivery opening.

Example 15: The cannula of any of examples 1 to 14, where the oxygen inlet is laterally offset from a center of the distribution section in a direction along the elongated oxygen delivery opening.

Example 16: The cannula of any of examples 1 to 15, where the distribution section is wedge-shaped.

Example 17: The cannula of any of examples 1 to 16, further including a diverging supply conduit between the oxygen inlet and the distribution section.

Example 18: The cannula of any of examples 1 to 17, where the at least one elongated oxygen delivery opening defines a pair of opposed elongated edges having different lengths.

Example 19: The cannula of any of examples 1 to 18, where the cannula body extends along a longitudinal axis, where the at least one elongated oxygen delivery opening includes at least two elongated oxygen delivery openings extending along the longitudinal axis and spaced from each other along the longitudinal axis, and where the distribution section includes a plurality of oxygen conduits, each oxygen conduit of the plurality of oxygen conduits fluidically coupling a respective elongated oxygen delivery opening to the oxygen inlet.

Example 20: The cannula of any of examples 1 to 18, where the cannula body extends along a longitudinal axis, where the at least one elongated oxygen delivery opening includes at least two elongated oxygen delivery openings extending transverse to the longitudinal axis and spaced from each other along the longitudinal axis, and where the distribution section includes a plurality of oxygen conduits, each oxygen conduit of the plurality of oxygen conduits fluidically coupling a respective elongated oxygen delivery opening to the oxygen inlet.

Example 21: The cannula of any of examples 1 to 20, where the cannula body further defines an oxygen vortex between the oxygen inlet and the distribution section.

Example 22: The cannula of any of examples 1 to 21, further including a mouthpiece extending from the cannula body and fluidically coupled to the exhalation lumen.

Example 23: A cannula including: a cannula body defining: an exhalation lumen configured to receive a volume of exhalation from a patient; an oxygen inlet at a surface of the cannula body; a plurality of elongated oxygen delivery openings fluidically coupled to the oxygen inlet and being arranged and sized to deliver substantially symmetrical oxygen flow to nostrils of the patient.

Example 24: The cannula of example 23, where each elongated oxygen delivery opening of the plurality of elongated oxygen delivery openings defines an opening periphery along the surface, and where the opening periphery is one of rectangular, rounded rectangular, n-sided polygonal, piecewise linear, piecewise curved, or curved.

Example 25: The cannula of examples 23 or 24, where the oxygen inlet is located along an end or along a corner of the cannula body.

Example 26: A system including: a capnography analysis module; an oxygen supply; and the cannula of any of examples 1 to 25 fluidically coupled to the capnography analysis module to deliver the volume of exhalation to the capnography analysis module and fluidically coupled to the oxygen supply to deliver oxygen to nostrils of the patient.

Example 27: A method of using any of the cannulas of examples 1 to 26.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device. These and other examples are within the scope of the following claims.

What is claimed is:

1. A cannula comprising:
a cannula body defining:
an exhalation lumen configured to receive a volume of exhalation from a patient;
an oxygen inlet at a surface of the cannula body; and
at least one oxygen delivery opening configured to deliver oxygen to nostrils of the patient, the at least one oxygen delivery opening extending along a surface of the cannula body and fluidically coupled to the oxygen inlet by a distribution section defined by the cannula body, the distribution section having a major cross-sectional area greater than a major cross-sectional area of the oxygen inlet.

2. The cannula of claim 1, wherein the at least one oxygen delivery opening comprises a pair of elongated oxygen delivery openings.

3. The cannula of claim 1, wherein the at least one oxygen delivery opening defines an opening periphery along the surface, and wherein the opening periphery is one of rectangular, rounded rectangular, n-sided polygonal, piecewise linear, circular, oval, ovoid, piecewise curved, or curved.

4. The cannula of claim 1, further comprising at least one prong extending from the surface of the cannula body.

5. The cannula of claim 4, wherein the at least one prong comprises a pair of prongs positioned along a line parallel to the at least one oxygen delivery opening, and wherein the prongs flank the oxygen delivery opening.

6. The cannula of claim 5, wherein the at least one oxygen delivery opening extends from a first opening end spaced from a first prong of the pair of prongs to a second opening end spaced from a second prong of the pair of prongs.

7. The cannula of claim 1, wherein the distribution section comprises at least one of a pair of opposed triangular walls, a pair of opposed rectangular walls, a pair of opposed boat-shaped walls, an angled wall opposing a flat wall, or a pair of opposed angled walls.

8. The cannula of claim 1, wherein the oxygen inlet is located along an end or along a corner of the cannula body.

9. The cannula of claim 1, wherein the distribution section is wedge-shaped.

10. The cannula of claim 1, wherein the cannula body extends along a longitudinal axis, wherein the at least one oxygen delivery opening comprises at least two oxygen delivery openings extending along the longitudinal axis and spaced from each other along the longitudinal axis, and wherein the distribution section comprises a plurality of oxygen conduits, each oxygen conduit of the plurality of oxygen conduits fluidically coupling a respective oxygen delivery opening to the oxygen inlet.

11. The cannula of claim 10, wherein the at least two oxygen delivery openings comprises at least two circular oxygen delivery openings, and wherein a first circular oxygen delivery opening of the at least two circular oxygen delivery openings comprises a first diameter and a second circular oxygen delivery opening of the at least two circular oxygen delivery openings comprises a second diameter, the first diameter being greater than the second diameter.

12. The cannula of claim 11, wherein the first circular oxygen delivery opening is positioned proximate to the oxygen inlet and the second circular oxygen delivery opening is positioned distal to the oxygen inlet.

13. The cannula of claim 1, wherein the cannula body further defines an oxygen vortex between the oxygen inlet and the distribution section.

14. The cannula of claim 1, further comprising a mouthpiece extending from the cannula body and fluidically coupled to the exhalation lumen.

15. A cannula comprising:
a cannula body defining:
an exhalation lumen configured to receive a volume of exhalation from a patient;
an oxygen inlet at a surface of the cannula body; and
a plurality of oxygen delivery openings fluidically coupled to the oxygen inlet and being arranged and sized to deliver substantially symmetrical oxygen flow to nostrils of the patient.

16. The cannula of claim 15, wherein each oxygen delivery opening of the plurality of oxygen delivery openings defines an opening periphery along the surface, and wherein the opening periphery is one of rectangular, rounded rectangular, n-sided polygonal, circular, oval, ovoid, piecewise linear, piecewise curved, or curved.

17. The cannula of claim 15, wherein the cannula body receives a volume of exhalation from a patient and delivers a volume of oxygen to nostrils of the patient, and wherein the cannula body is fluidly coupled to and receives the volume of oxygen from an oxygen supply and is fluidly coupled to a capnography analysis module to deliver the volume of exhalation to the capnography analysis module.

18. The cannula of claim 15, wherein the plurality of oxygen delivery openings are fluidly coupled to the oxygen inlet by a distribution section defined by the cannula body, and wherein the plurality of oxygen delivery openings comprise a first circular oxygen delivery opening positioned along the distribution section at a first distance from the oxygen inlet and a second circular oxygen delivery opening positioned along the distribution section at a second distance from the oxygen inlet, the first distance being less than the second distance.

19. The cannula of claim 18, wherein a first diameter of the first circular oxygen delivery opening is greater than a second diameter of the second circular oxygen delivery opening.
